(19) United States
(12) Patent Application Publication
(10) Pub. No.: US 2011/0034827 A1
(43) Pub. Date: Feb. 10, 2011

(54) METHOD AND A DEVICE FOR HEARING-RELATED EXAMINATION

(75) Inventor: Leif Rix, Lund (SE)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: Entomed AB, Malmo (SE)

(21) Appl. No.: 12/297,514

(22) PCT Filed: Apr. 17, 2007
(86) PCT No.: PCT/SE2007/000356

§ 371 Date: Sep. 8, 2009

(30) Foreign Application Priority Data
Apr. 19, 2006 (SE) 0600851 0

(51) Int. Cl. A61B 5/12

(52) U.S. Cl. 600/559

(57) ABSTRACT

Method for hearing-related examination comprising the steps of generating sound signals and/or electrical signals in an examination unit (12). The method includes the steps of removably connecting the examination unit (12) as a modular unit to a base unit (11) and removably connecting a function card (13) to the base unit (11). Further steps are sending control signals from the function card (13) to the examination/module unit (12) for performing a specific examination, and obtaining test result data in the examination unit (12) and collecting said test result data to the function card for storage therein. The device comprises a base unit (11) having a display (15), input means (16; 17; 45) and output means (15) for performing the examination. The base unit (11) comprises a first interface (14) receiving a function card (13) and the function card (13) comprises a first control unit (33), a data memory (31) and a program memory (32) a, said program memory storing software for performing an examination and said data memory (31) storing data related to the examination. The base unit (11) comprises a second in-terface (28) receiving at least one modular unit (12) having a micro controller (29), examination output and input means, and the data memory (31) of the function card is connected to said micro controller (29) of the modular unit for collecting and storing examination data.

### TECHNICAL FIELD

Audiometers and other equipment for hearing-related examination and analysis are used throughout the world in different applications. Comparatively simple audiometers are used in screening tests where individuals of a group are tested for hearing losses. In general audiometry is concerned with an individual's sensitivity or tolerance. Other aspects of audiometry are to determine discrimination levels, the ability to distinguish speech from background noise and the ability to recognize pitch.

In a clinical environment very detailed medical examinations are performed, normally with specifically designed and more sophisticated audiometers. Other forms of hearing-losses are measured and examined in a process called tympanometry or by measuring otoacoustic emission (OAE).

Tympanometry is a measurement of acoustic immittance of the ear canal and middle ear. Acoustic immittance is a general term used to refer either to acoustic impedance or acoustic admittance measurements. At present most of the devices are based on admittance measurement. Acoustic admittance is the ease with which acoustic energy is transferred from one system to another, which is the opposite of acoustic impedance. If the air in the ear canal is easily set into a vibration, the admittance is high. If the air is difficult to set into a vibration, the admittance of the system is low. Tympanometry is the measurement of acoustic admittance as a function of ear canal pressure and the resulting graph is a tympanogram. Positive or negative pressure introduced to the sealed ear canal decreases the admittance of the air in the ear canal by stiffening the tympanic membrane. The effect of air pressure on acoustic admittance measured in the ear canal is systematically altered by the middle ear disease.

The primary purpose of otoacoustic emission (OAE) tests is to determine cochlear status, specifically hair cell function. The normal cochlea does not just receive sound; it also produces low-intensity sounds called OAEs. The information can be used to (1) screen hearing (particularly in neonates), (2) partially estimate hearing sensitivity within a limited range, (3) differentiate between the sensory and neural components of sensorineural hearing loss, and (4) test for functional (feigned) hearing loss. The information can be obtained from patients who are sleeping or even comatose because no behavioral response is required. Four types of otoacoustic emissions are considered:

Spontaneous otoacoustic emissions (SOAEs)—Sounds emitted without an acoustic stimulus (ie, spontaneously)

Transient otoacoustic emissions (TOAEs) or transient evoked otoacoustic emissions (TEOAEs)—Sounds emitted in response to an acoustic, stimuli of very short duration; usually clicks but can be tone-bursts

Distortion product otoacoustic emissions (DPOAEs)—Sounds emitted in response to 2 simultaneous tones of different frequencies

Sustained-frequency otoacoustic emissions (SFOAEs)—Sounds emitted in response to a continuous tone

### PRIOR ART

Various systems for measuring auditory responses are previously known, and prior art audiometer systems normally are designed as stand-alone units. Such systems are referred to as audiometers, and usually provide for application of selected tones, broad-band noise, and narrow-band noise variable in frequency and amplitude, respectively, to aid in determining the amount of hearing loss a person may have. An audiometer presents a variety of stimuli under strict frequency, temporal and level control to persons for the purpose of testing their hearing capability. Specifications for controlling these parameters are provided in ANSI Standard S3.6.

To assess hearing thresholds for speech, an audiometer may also reproduce live voice or recorded speech at selectable calibrated levels. Various complex controls are used to administer varying sound conditions to determine a range of responses for the individual. These responses can be charted or graphed, and can serve as the basis for applying programming signals to programmable hearing aids. Also tympanometers used in tympanometry and devices used for otoacoustic emission (OAE) tests normally are designed as stand-alone devices. A more developed system for hearing-related analysis is disclosed in US2004/0204921. This system is a portable system using a host computer with standardized ports and a hearing aid programming card used with one port and a hearing-related analyzer card used with the other port. All input and output devices such as microphones, loudspeakers and switches are connected to the cards. The system in accordance with US2004/0204921 is used to program a hearing aid device based on an input from an audiometer card.

Stand-alone devices used in the above mentioned applications are specifically designed for each type of test or examination. On one hand this is an advantage since the device normally performs very well in specified tasks. However, in other aspects it is a disadvantage since different devices have to be used when different examinations are performed. In a smaller clinic or nursing home it will be considered a drawback that each device has a limited area of operation.

Tests and examinations are the subject of development and altered requirements. In conventional audiometers and similar devices some changes of performance or process will require an exchange of basic units or the complete device. A simplified way of updating the device would be highly desirable.

### SUMMARY OF THE INVENTION

An object of present invention is to overcome the drawbacks and disadvantages set out above and to provide a method and a device for hearing-related examination that are more flexible and more easily adapted to new examination regulations and new examination methods.

A further object of the invention is to improve the possibilities for documentation of examinations, the calibration of the device and also a more individualized management of the device.

In accordance with the invention the device is separated in three different sections. A first section is a human machine interface or base unit comprising a case, a graphical or character display and control buttons or knobs. The base unit can have an appearance similar to a prior art audiometer but will be operated differently.

Most or all of the hardware used during examination is included in a separate modular unit that is received in the case of the base unit and readily replaced or completed with other modules for different tests or examinations. The hardware in the modular unit comprises memory units, a micro control unit, I/O control units, and specific units for performing each type of test, such as a tone generator and a noise generator. The modular units are connected to a first interface including a bus system in the base unit.

The base unit includes also a second interface for connecting to a function card. The function card comprises all necessary software and control functions for performing tests and examinations. The software is stored in a program memory unit. Different data, such as data regarding test results, device information, and information relating to the personnel performing the test are stored in a data memory unit. The graphical device and associated I/O units of the base unit can be modified in accordance with control signals from the function card and will thus be adapted to each specific test or examination.

In accordance with the invention the base unit is a general or universal device that is utilized in each specific application through the specific function card and the specific module unit that are used together with the base unit. When a test or an examination is to be performed the person responsible for the test inserts an appropriate function card in the base unit. The software and control function of the function card set up the base unit and the module unit, so as to be able to execute the specific examination.

The base unit comprises a keyboard and a set of knobs for manually performing an examination and for controlling the modular unit. A display is included in the base unit for displaying information related to initiation and operation of the modular unit and also to authorizing personnel and to the performance of the examination. The display can be altered by the function card depending on the test that is executed. The base unit also comprises hardware for communicating with other external units, such as printers, computers and telephone equipment.

In the function card a variety of information is stored. The information can include settings and test results. Normally, a specific function card is associated to a specific modular unit, so as to set the complete device into an operational mode for performing a specific examination. In accordance with one aspect of the invention the function card is personal and includes specific personal information and personal settings relating to language and individual authority. The function card can also include information related to the payment and reimbursement for the examination.

### BRIEF DESCRIPTION OF THE DRAWINGS

These and other more detailed features and an understanding of the invention will become apparent from a consideration of the following detailed description of different embodiments thereof in view of the drawings.

FIG. 1 is a diagrammatic view of one embodiment of the invention showing a function card, a base unit and four modular units.

FIG. 2 is a diagrammatic view of one embodiment of the function card in FIG. 1

FIG. 3 is a diagrammatic view of one general embodiment of the modular units in FIG. 1.

FIG. 4 is a perspective view of one embodiment of the invention.

### DETAILED DESCRIPTION

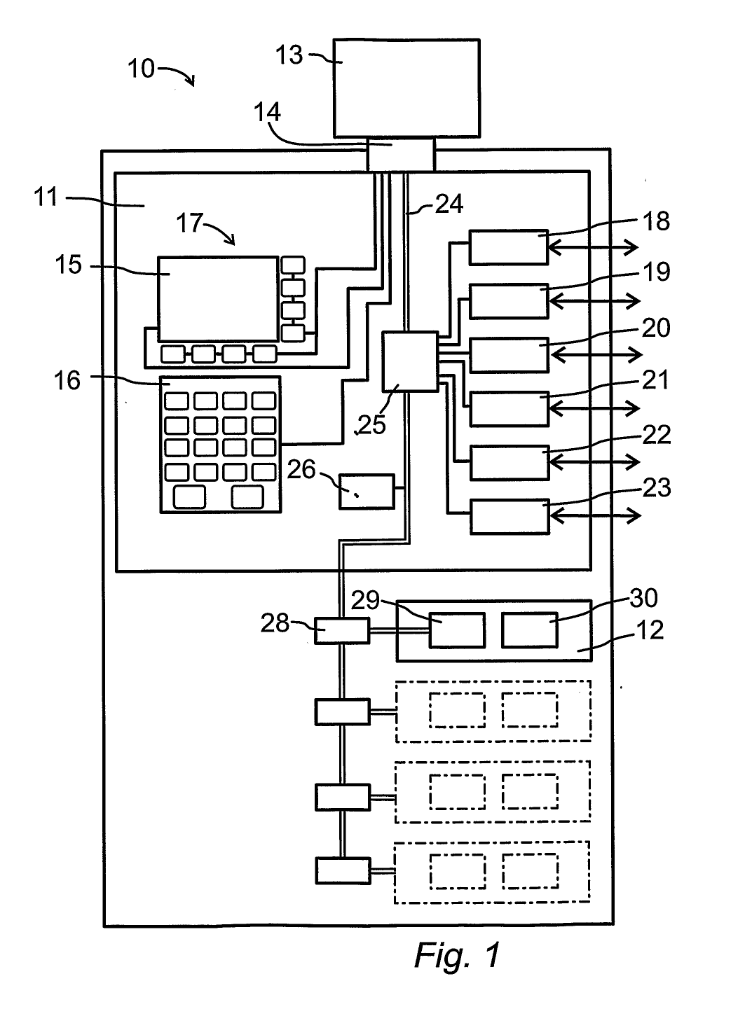

In FIG. 1 one embodiment of a device 10 in accordance with the invention includes a base unit 11 with a casing, a first modular unit 12 and a function card 13. The function card 13 is connected to the base unit 11 through a fist interface 14. The base unit 11 comprises input-output means, such as a display 15, a keyboard 16 and a set of buttons 17, some of which are arranged along side edges of the display 15. Further buttons and controls can be arranged on the base unit 11 for specific purposes, c.f. FIG. 4. The set of buttons 17 can be replaced or completed with a pressure sensitive display unit.

The base unit 11 is used an interface between persons involved with the examination or test, the function card 13, modular units 12 and external equipment, such as printers, servers and telephone equipment. In the embodiment shown in FIG. 1 the interface comprises a RS-232 communications means 18, a USB communication means 19, a LAN/WLAN communications means 20, a BLUETOOTH communications means 21, a GSM communications means 22 and a GPRS communications means 23. A bus 24 connects the first interface 14 to different parts of the base unit 11.

Each of the communication means 18-23 is connected to a digital switch 25 allowing anyone of them to be connected to the bus 24 and to other units. The base unit 11 also comprises a real time clock circuit 26 connected to electronic parts of the base unit, such as the input-output means and the display.

The first modular unit 12 functions as an examination unit and is arranged within the casing of the base unit 11 in the shown embodiment and is connected to the bus 24 through a second interface 28. Most functions of the modular unit 12 can be controlled by the function card 13 through the bus. Each modular unit comprises a micro controller 29 and a memory 30 and hardware specifically designed for a specific type of examination, such as audiometry, tympanometry or OAE. In accordance with the invention a plurality of modular units can be received in the base unit.

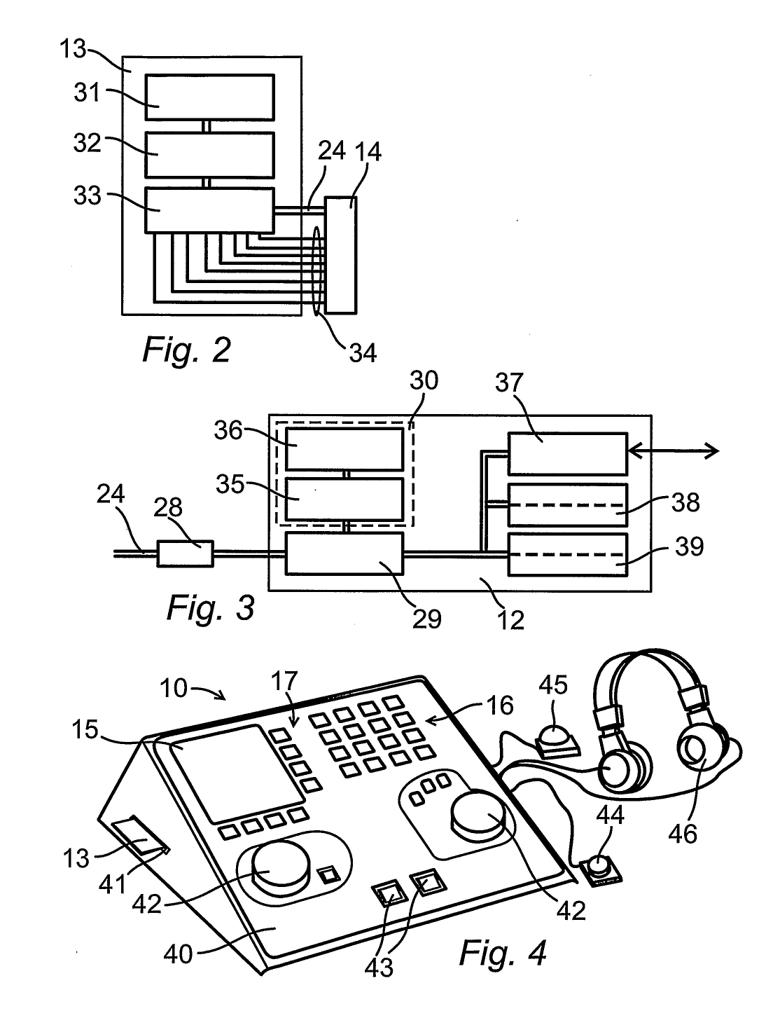

As set out above most control functions of the device are gathered in the function card 13. In the embodiment shown in FIG. 2 the function card comprises a memory divided into a data memory 31 and a program memory 32, and a control unit 33. Basically, the data memory 31 stores settings and data related to specific examinations and users. The settings include settings of the base unit 11, such as the function and appearance of the display and input-output means, and settings of the modular units 12 that are included in or connected to the base unit. Normally a function card 13 is associated to one or a limited number of modular units 12, so as to be able to set up and control relevant functions of the modular units.

When the function card 13 is used as a personal card for an operator of the device a plurality of settings related to language, authority and other personal requirements is stored in the data memory 31. As a result the display 15 and other input-output means will be personalized which will facilitate examinations and lower the chances of errors in connection with the examinations. Naturally a plurality of function card associated to different persons and/or to different personnel functions can be used with and control a specific modular unit 12 and a specific base unit 11.

The data memory 31 also stores examination results and other documentation related to examinations. These data can transferred to central servers or similar means at a later stage if appropriate and if they are not transferred directly by the communication means of the base unit.

A plurality of hardware settings of the modular units specify the basic functions, capacity and performance of each modular unit, and most of these settings can be controlled by the function card through the bus 24. Among these basic functions are frequency intervals, output levels and input sensitivity.

The steps of and performance of each type of examination are controlled by the content of the program memory 32 of the function card. As a result any changes in the requirements of an examination readily can be incorporated in the device by updating or replacing the function card or the content of the program memory 32. Examination results are collected from the modular unit 12 to the data memory 31 and further on to external devices through a suitable communication means, such as the USB communication means 19.

Besides basic examination data also data relating to the version and identity of the modular unit 12, the software and data in the program memory 32 and data memory 31 and the base unit 11 can be stored in the data memory 31 and/or transferred to external devices. In this way it will be possible to document and verify examinations and performance of the device. Also data related to calibration of the device, including the modular units, and temperature and other ambient conditions during examination are retrieved and included in the documentation.

According to a further aspect of the invention the function card 13 also includes features for limiting and controlling the use of the device. In one embodiment the function card 13 is designed to allow a limited number of examinations to be performed. After completion of this number of examinations the function card has to be updated or replaced to allow further tests and examinations to be carried out. This function could easily be implemented by including in the program memory 32 of the function card a set of instructions requesting the user to input a code to “unlock” or load the device with a further set of examinations. The code can be accessed through the Internet or any other suitable way after payment of a fee. This feature will allow a user to pay not for the device as such but for the actual use of it.

The function card 13 shown in FIG. 2 comprises also a section of the first interface 14 allowing the function card to communicate with the base unit 11 and any connected modular unit 12. The first interface 14 includes a section of the bus 24 and a plurality of control lines 34 that are connected to different sections and circuits of the base unit. The function card 13 controls all essential functions of the base unit 11 and any connected modular unit 12 by sending instructions and receiving data through the bus 24 and the control lines 34.

Each of the modular units 12 is connected to the base unit 11 through the second interface 28 as shown in FIG. 3. Preferably the base unit 11 comprises a casing capable of receiving at least two modular units 12, c.f. FIG. 4. Each modular unit 12 comprises a memory 30 divided into a module program memory 35 and a module data memory 36 and a micro controller 29. Depending on the basic functions of the modular units further circuits are included. In the embodiment shown in FIG. 3 and designed for audiometry an I/O control unit 37 is included for transmitting and receiving electronic and audio signals. External hardware, such as earphones, loudspeakers, transducers and switches, is connected to the modular unit 12 through the I/O control unit 37.

The signals that are used during an examination are generated in a tone generator 38 and a noise generator 39. In a preferred embodiment each generator is divided into two sections, one for each ear. The tone generator 38 is capable of generating pure tone waves and frequency modulated pure tone waves. The noise generator 39 generates a narrow band white noise initially which is filtered into speech-spectrum noise. The micro controller 29 controls on the basis of the content in the module program memory 35 and on instructions received from the function card 13 through the bus 24 most functions and circuits of the modular unit 12. In a conventional manner the modular unit 12 comprises also attenuators and filters, so as to be able to produce any required frequency at any required level.

An example of a physical embodiment of the device in accordance with the invention is shown in FIG. 4. In this embodiment a casing 40 similar to a casing used for a conventional audiometer is used to encompass the circuitry of the base unit 11 and a modular unit 12. The function card 13 is shown partly inserted in a slot 41 of the casing 40 where it is connected to the first interface 14 (not shown). In a front or upper side of the casing the display 15, keyboard 16 and set of buttons are arranged to be readily accessible by the personnel performing the examination.

Further knobs 42 and switches 43 are provided in a conventional manner to facilitate each type of examination. The function of the knobs 42 and switches 43 as well as of the display 15, keyboard 16 and buttons 17 can be altered by the function card, so as to be adapted to the specific examination that is to be performed. Some controls, such as a power on and power off switch, normally are fixed and not depending on the function card or specific examination.

The embodiment of the device shown in FIG. 4 also includes a response button 44 that is used by a test person during a test, such as a conventional audiometry examination. The response button 44 is connected to the modular unit 12. Other modular units designed for other types of examinations include other types of response devices, such as microphones, pressure transducers and electrical sensors.

A break or stoppage of a current examination can be achieved by depressing or otherwise activating an emergency knob 45. Normally the emergency knob 45 is connected to or is a part of the base unit 11 and will interrupt any on-going process, irrespective of the modular unit presently being used. For an audiometry examination a pair of headphones 46 is used as output means.

1. Method for hearing-related examination comprising the steps of generating sound signals and/or electrical signals in an examination unit (12), characterised by the steps of
removably connecting the examination unit (12) as a modular unit to a base unit (11),
removably connecting a function card (13) to the base unit (11) sending control signals from the function card (13) to the examination/module unit (12) for performing a specific examination, and
obtaining test result data in the examination unit (12) and collecting said test result data to the function card for storage therein.

2. Method in accordance with claim 1, further including the step of displaying examination related data on a display (15) of the base unit (11).

3. Method in accordance with claim 1, further including the step of receiving control input signals from input means (16; 17; 42; 43) of the base unit (11) and transferring the control input signals to the function card (13).

4. Method in accordance with claim 1, further including the step of connecting the function card (13) to the base unit (11) through a bus (24).

5. Method in accordance with claim 4, further including the step of sending the control signals from the function card (13) to the examination unit (12) through the bus (24).

6. Method in accordance with claim 1, further including the steps of counting in the function card (13) each time a specific examination is performed and limiting the number of examination performances to an adjustable number.

7. A device for hearing-related examination comprising a base unit (11) having a display (15), input means (16; 17; 45) and output means (15) for performing the examination, characterised in
that the base unit (11)comprises a first interface (14) receiving a function card (13),
that the function card (13) comprises a first control unit (33), a data memory (31) and a program memory (32) a, said program memory storing software for performing an examination and said data memory (31) storing data related to the examination,
that the base unit (11) comprises a second interface (28) receiving at least one modular unit (12) having a micro controller (29), examination output and input means, and
that the data memory (31) of the function card is connected to said micro controller (29) of the modular unit for collecting and storing examination data.

8. A device in accordance with claim 7, wherein said first interface (14) comprises a bus (24) and a plurality of control lines (34) allowing said control unit to interact with said at least one modular unit.

9. A device in accordance with claim 8, wherein the bus (24) is extended through the second interface (28).

10. A device in accordance with claim 7, wherein the base unit (11) is provided means (18; 19; 20; 21; 22; 23) for communicating with external units.

11. A device in accordance with claim 10, wherein said means are connected to a digital switch (25).
